(19) United States
(12) Patent Application Publication
(10) Pub. No.: US 2021/0128231 A1
(43) Pub. Date: May 6, 2021

(54) ENERGY TRANSMITTING THERAPEUTIC MEDICAL DEVICE

(71) Applicant: Medwaves, Inc., San Diego, CA (US)

(72) Inventors: Theodore Ormsby, San Diego, CA (US); George Leung, San Diego, CA (US); Gwo Jenn Shen, San Diego, CA (US)

(21) Appl. No.: 17/083,751

(22) Filed: Oct. 29, 2020

Related U.S. Application Data
(60) Provisional application No. 62/930,107, filed on Nov. 4, 2019.

(51) Int. Cl. A61B 18/14

(52) U.S. Cl. CPC A61B 18/1492; A61B 2018/00964

(57) ABSTRACT

An energy-transmitting therapeutic device having a hollow elongated assembly with a flexible tip at its distal end in which the hollow elongated assembly includes energy-transmitting elements and directional control elements. The directional control elements control the flexible tip such that it can pass through a lumen having a curve with a minimum radius of 1 cm and the energy-transmitting elements can deliver energy at an electromagnetic frequency of 300 KHz to 300 GHz.

### CROSS-REFERENCE TO RELATED APPLICATIONS

The present application claims the benefit of U.S. Provisional Patent Application Ser. No. 62/930,107, filed on Nov. 4, 2019. The content of this prior application is hereby incorporated by reference in its entirety.

### BACKGROUND

Medical devices that deliver energy for therapeutics are used to treat a variety of diseases and conditions in the human body. Such devices must be flexible enough to negotiate twists and turns of guiding medical device lumens, such as endoscopes and catheters, as well as those of biological structure lumens, e.g., blood vessels and bronchial cavities.

There is a need to develop devices that have the ability to maintain certain geometries and curvatures after negotiating twists and turns while being advanced through a lumen and still have the capacity for directional control to position the device within the diseased volume of a targeted tissue.

### SUMMARY

An energy-transmitting therapeutic device is disclosed meeting the need described above. The device includes a hollow elongated assembly having one end configured for attachment to a therapeutic energy-delivering catheter and the other end having a flexible tip that contains one or more energy-transmitting elements. The hollow elongated assembly includes electrical conductors electrically connected to the energy-transmitting elements and directional control elements located along the assembly. The directional control elements control the geometry of the flexible tip such that it can pass through a lumen having a curve with a minimum radius of 1 cm, and transmission impedance and impedance transition of the energy-transmitting elements are stable and consistent within the geometry of the flexible tip. The energy-transmitting elements deliver energy at an electromagnetic frequency of 300 KHz to 300 GHz.

The details of the invention are set forth in the description below. Other features, objects, and advantages of the invention will be apparent from the detailed description and the drawings below, and also from the appending claims.

### BRIEF DESCRIPTION OF THE DRAWINGS

FIG. 1: Exemplary use of directional control elements to position the hollow elongated assembly, i.e., therapeutic energy delivery element with directional control.

FIG. 2: Examples of direction control element shapes.

FIG. 3: Exemplary arrangement of elements for an energy transmitting therapeutic medical device.

### DETAILED DESCRIPTION

The hollow elongated assembly of this invention, as summarized above, includes directional control elements located along the assembly. The directional control elements control the geometry of the flexible tip such that the tip can pass through a lumen having a curve with a minimum radius of 1 cm. The directional control elements can also control the geometry and curvature of the entire hollow elongated assembly to facilitate advancing the assembly through an irregularly-shaped lumen of a guide device or a biological tissue.

The directional control elements can be active elements or passive elements.

Active elements generate forces by mechanical, electrical, thermal, hydraulic, or chemical means. For example, mechanical means can be uni- or bi-directional pull wires, screws, and gears. In another example, electrical or thermal means can be an electrically or thermally affected bimetal. Thermal means can also be a thermal memory metal. Turning to hydraulic means, this can be a hydraulically-activated elastic cavity, e.g., a balloon.

As to chemical means, it can be chemically-induced elongation or compression. In a particular example, chemically-induced morphing, i.e., shape changing, of polyurethane shape memory polymer (SMP) micro fibers and/or springs generates force in the claimed invention. In a specific application of this example, the presence of water and/or ethanol releases residual strain/stress captured during the fabrication process of the SMP. This release serves as the driving force for morphing, e.g., self-winding and self-twisting/untwisting.

The directional control elements can include a combination of mechanically, electrically, thermally, hydraulically, and chemically-activated active elements to affect the geometry of the hollow elongated assembly as needed. In one example, the active elements generate forces by electro-mechanical, electro-thermal, and electro-chemical means.

As mentioned above, the directional control elements can be passive elements.

An exemplary passive element is formed of a memory material. The memory material can be a spring metal including, but not limited to, steel, nickel, copper, titanium, beryllium, or an alloy of these metals, e.g., nitinol.

An alternative passive element is formed of a dielectric polymer. The dielectric polymer can be, e.g., polytetrafluoroethylene, polycarbonate, nylon, polyether ether ketone, silicone rubber, polyurethane, and polyethylene.

Any of the directional control elements set forth above that are passive elements can be rectangular, oval, half-oval, half-tubular, triangular, or trapezoidal in cross-section to provide a desired biased directional control. For example, a directional control element having a triangular cross-section will be stiffer at the apexes of the triangle and will tend to resist flexing at these points, as compared to points along the sides of the triangular shape.

In an aspect of the invention, the directional control elements can include both active elements and passive elements to enhance directional control positioning of the flexible tip into a target biological tissue, e.g., a tumor. For example, during advancement of the energy-transmitting therapeutic device through an irregularly-shaped lumen, active elements can be operated to curve the device so that it can negotiate a curve in the lumen. After the device is successfully advanced around a curve, the passive elements can return the device to its original shape, e.g., straight, so that it can be further advanced through the lumen.

Repeating from the SUMMARY section above, the energy-transmitting therapeutic device of the invention includes energy transmission elements at the flexible tip of the hollow elongated assembly. The energy-transmitting elements can be, for example, a transmission line and an antenna system for transmitting an electromagnetic field into a biological tissue in substantial contact with the flexible tip. The energy-transmitting elements deliver energy at an electromagnetic frequency of 300 KHz to 300 GHz. The energy-transmitting elements are electrically connected to electrical conductors. During operation of the device, the electrical conductors are also electrically connected to a power supply that provides the electromagnetic energy.

The above device is designed such that transmission impedance and impedance transition of the energy-transmitting elements are stable and consistent within the geometry of the flexible tip.

This design eliminates issues such as interruption and attenuation of the transmission of the electromagnetic field in the above frequency range by irregular impedance along the electrical path from changes in inner and outer conductor physical relationship. Also avoided is a diminution of emission field patterns and efficiency of antennas due to deformation of its elements and their physical relationship. Moreover, as the antenna transforms electric current into an electromagnetic field and couples it to the surrounding environment, its ability to maintain and recover structural integrity is important for it performing as design. Additionally, a transition connection from coaxial transmission line to antenna elements is designed to be stable and consistent for the same reasons.

In an exemplary device, the directional control elements are part of or form the energy transmission elements. In this device, a portion of the directional control elements is formed of a memory material that is a spring metal capable of transmitting and transforming electromagnetic power. Another portion of the directional control elements is formed of a dielectric material.

Without further elaboration, it is believed that one skilled in the art can, based on the above description, utilize the present invention to its fullest extent. The following specific examples are, therefore, to be construed as merely illustrative, and not limitative of the remainder of the disclosure in any way whatsoever.

### EXAMPLES

### Example 1: Treatment of Lung Cancer with the Energy-Transmitting Therapeutic Device

In FIG. 1, an energy-transmitting therapeutic device of the invention is attached at the end of a catheter and advanced through a bronchoscope to a site, e.g., a tumor, in the lung. During the procedure, the device must be able to negotiate the curvature of the bronchoscope. When the catheter is fully advanced to the tumor site, the energy-transmitting therapeutic device of the invention is used to deliver to the tumor electromagnetic energy at the flexible tip of the hollow elongated assembly, thereby destroying cancer cells. The directional control elements can be used to reposition the flexible tip or the entire hollow elongated assembly to treat an adjacent area without withdrawing the catheter.

### Example 2: Cross-Sections of Directional Control Elements

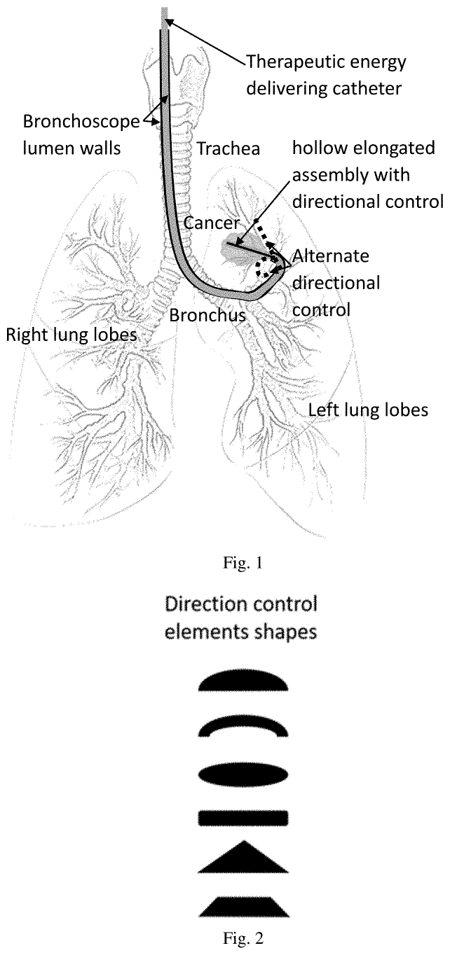

The directional control elements can have particular cross-sectional shapes depending upon the particular application and shape of the lumen through which the device must pass. Exemplary cross-sectional shapes are shown in FIG. 2. The stiffness of the directional control element in a particular direction is dictated, in part, by its cross-sectional shape. Each shape will confer on the directional control elements a preferred geometry in its relaxed state. In turn, in the absence of external forces, the elements will return the device to a particular geometry.

The elements can be solid or have a hollowed interior cavity. A directional control element having a hollowed interior cavity can be included in the device of the invention. In such a device, the directional control elements can be hydraulically-activated elastic cavities, i.e., active elements.

### Example 3: Internal Arrangement of an Exemplary Energy Transmitting Therapeutic Device

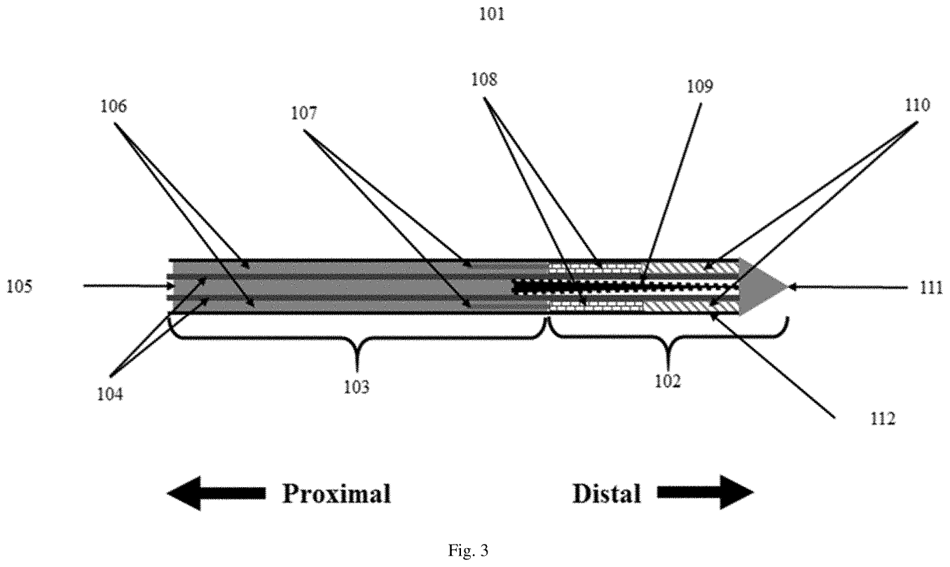

An exemplary hollow elongated assembly 101 of the energy transmitting therapeutic device is shown in FIG. 3. The energy transmitting therapeutic device can be used as part of a therapeutic energy-delivering catheter.

Hollow elongated assembly 101 has an energy emitting region 102 at its distal end and an energy transmitting region 103 at its proximal end.

Energy transmitting region 103 contains an inner coaxial conductor 105, an outer coaxial conductor 106, and dielectric insulation 104 located between inner coaxial conductor 105 and outer coaxial conductor 106. Outer coaxial conductor 106 extends substantially along the entire length of energy transmitting region 103. Inner coaxial conductor 105 can also extend substantially along the entire length of energy transmitting region 103 or, as shown in FIG. 3, it can extend to a point short of the entire length of energy transmitting region 103. A coaxial antenna choke 107 is located at the distal end of energy transmitting region 103. Coaxial antenna choke 107, which can be a single or double coaxial antenna choke, is embedded within outer coaxial conductor 106.

Energy emitting region 102 contains at its distal end a penetrating flexible antenna tip 111, which is sufficiently flexible to negotiate a turn having a radius of 1 cm in a lumen having a diameter of 2 mm. Also contained within energy emitting region 102 are transformational impedance matching dielectric layers 108 and a coaxial antenna tip-shaft 110 located just proximate to penetrating flexible antenna tip 111. Further, a transformational impedance matching inner coaxial electrical conductor 109 is located in the center of energy emitting region 102 and extends along the length of energy emitting region 102 up to penetrating flexible antenna tip 111. In hollow elongated assembly 101 shown in FIG. 3, transformational impedance matching inner coaxial electrical conductor 109 is in electrical communication with inner coaxial conductor 105 and extends from the distal end of energy transmitting region 103 to penetrating flexible antenna tip 111. Of note, transformational impedance matching dielectric layers 108 and coaxial antenna tip-shaft 110 are separated from transformational impedance matching inner coaxial electrical conductor 109 by dielectric insulation 104, which extends from energy transmitting region 103 through energy emitting region 102.

Importantly, transformational impedance matching inner coaxial electrical conductor 109 shown in FIG. 3 also serves as the directional control element described above. In other embodiments of hollow elongated assembly 101, transformational impedance matching inner coaxial electrical conductor 109 is distinct from and co-located with one or more directional control elements.

An exemplary hollow elongated assembly 101 includes directional control elements that are discontinuous along the length of hollow elongated assembly 101, yet electrical continuity in terms of transition and impedance are consistent and stable under changing directions and deflections.

An outer insulation layer 112 covers the length of hollow elongated assembly 101 excluding penetrating flexible antenna tip 111.

As mentioned above, the proximal end of hollow elongated assembly 101 is configured for attachment to a therapeutic energy-delivering catheter. When hollow elongated assembly 101 is attached to the therapeutic energy-delivering catheter, inner coaxial conductor 105 becomes electrically connected to electrical conductors, which, in turn, are also electrically connected to a power supply that provides electromagnetic energy.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further, from the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. An energy-transmitting therapeutic device, comprising a hollow elongated assembly having a proximal end and a distal end, the proximal end configured for attachment to a therapeutic energy-delivering catheter and the distal end including a flexible tip that contains one or more energy-transmitting elements, the hollow elongated assembly including:
electrical conductors extending from the proximal end and electrically connected to the one or more energy-transmitting elements; and
directional control elements located along the hollow elongated assembly from the proximal end to the distal end,
wherein the directional control elements are configured to control a geometry of the flexible tip such that it can pass through a lumen having a curve with a minimum radius of 1 cm and the one or more energy-transmitting elements are configured to deliver energy at an electromagnetic frequency of 300 KHz to 300 GHz and transmission impedance and impedance transition of the one or more energy-transmitting elements are stable and consistent within the geometry of the flexible tip.

2. The device of claim 1, wherein the directional control elements are active elements or passive elements that control the geometry of the hollow elongated assembly such that the hollow elongated assembly can negotiate irregularly-shaped lumens of guide devices or biological tissues.

3. The device of claim 2, wherein the directional control elements are active elements that generate forces by mechanical, electrical, thermal, hydraulic, or chemical means.

4. The device of claim 3, wherein the forces are generated by electro-mechanical, electro-thermal, and electro-chemical means.

5. The device of claim 2, wherein the directional control elements are passive elements formed of a memory material.

6. The device of claim 5, wherein the memory material is a spring metal capable of transmitting and transforming electromagnetic power.

7. The device of claim 6, wherein the spring metal is steel, nickel, copper, titanium, beryllium, or an alloy thereof.

8. The device of claim 2, wherein the directional control elements are passive elements formed of a dielectric polymer.

9. The device of claim 8, further comprising second directional control elements formed of a spring metal, wherein the directional control elements and the second directional control elements are part of the one or more energy transmission elements.

10. The device of claim 9, wherein the energy transmission elements are configured as a transmission line and an antenna system for transmitting an electromagnetic field into a biological tissue in substantial contact with the flexible tip.

11. The device of claim 1, wherein the directional control elements include both active elements and passive elements to enhance directional control positioning of the flexible tip into a desired biological tissue.

12. The device of claim 1, wherein the directional control elements are rectangular, oval, half-oval, half-tubular, triangular, or trapezoidal in cross-section to provide a desired biased directional control.

13. The device of claim 2, wherein the directional control elements include both active elements and passive elements to enhance directional control positioning of the flexible tip into a desired biological tissue.

14. The device of claim 2, wherein the directional control elements are rectangular, oval, half-oval, half-tubular, triangular, or trapezoidal in cross-section to provide a desired biased directional control.
